(19) United States
(12) Patent Application Publication
(10) Pub. No.: US 2017/0074376 A1
(43) Pub. Date: Mar. 16, 2017

(54) MANIPULATOR WITH LINEAR MOTION ACTUATOR

(71) Applicant: EMPIRE TECHNOLOGY DEVELOPMENT LLC, Wilmington, DE (US)

(72) Inventor: Masaaki MIHARA, Chiba-shi (JP)

(73) Assignee: EMPIRE TECHNOLOGY DEVELOPMENT LLC, Wilmington, DE (US)

(21) Appl. No.: 15/309,179

(22) PCT Filed: May 5, 2014
(86) PCT No.: PCT/US14/36830

§ 371 Date: Nov. 4, 2016

(51) Int. Cl. F16H 25/22; A61B 1/018; F16H 19/04; A61B 17/00; A61B 1/00; F16H 25/20; A61B 34/00; A61B 1/313

(52) U.S. Cl. CPC F16H 25/2204; A61B 34/70; A61B 1/018; A61B 1/3132; A61B 17/00234; A61B 1/00098; F16H 25/2015; F16H 19/04; A61B 2034/2059

(57) ABSTRACT

A linear motion actuator is disclosed. The linear motion actuator can include a double-shafted motor having a first motor shaft with a first screw thread and a second motor shaft with a second screw thread. The first screw thread can have certain characteristics that are different from that of the second screw thread. The linear motion actuator is disclosed for use in an arm assembly of a laparoscopic manipulator, but can also be used in other types of devices.

### BACKGROUND

Field of the Invention

Embodiments disclosed herein relate generally to manipulators and linear motion actuators that may be employed in a manipulator or elsewhere. In particular, the manipulator may be used in laparoscopic surgery.

Description of the Related Art

Manipulators of various types are used in laparoscopic surgery. In laparoscopic or minimally invasive surgery operations are performed through one or more small incision. There can be a number of advantages to the patient with laparoscopic surgery versus an open procedure. These can include reduced pain due to smaller incisions and hemorrhaging, and shorter recovery time. At the same time, laparoscopic surgery is limited in that the small incisions only allow relatively small devices into the body and once there, there is limited space within the body. Because of these limitations and many more considerations, there is a continual need for improvement in devices, such as manipulators, for use in laparoscopic surgery.

### SUMMARY

A linear motion actuator is disclosed. The linear motion actuator can include a double-shafted motor having a first motor shaft with a first screw thread and a second motor shaft with a second screw thread. The first screw thread can have certain characteristics that are different from that of the second screw thread. For example the pitch and/or lead of the first screw thread can be different from that of the second screw thread. In addition, the pitch of the first screw thread can be inverse to the pitch of the second screw thread. The linear motion actuator is disclosed for use in an arm assembly of a laparoscopic manipulator, but can also be used in other types of devices.

According to some embodiments, a linear motion actuator can include a double-shafted motor, and first and second shaft guides. The double-shafted motor can comprise a first motor shaft having a first screw thread and a second motor shaft having a second screw thread. A pitch of the first screw thread can be different from that of the second screw thread. The first shaft guide can have a threaded hole which engages with the first screw thread. The second shaft guide can have a threaded hole which engages with the second screw thread.

The linear motion actuator may further include a housing to accommodate the motor, the first shaft guide, and the second shaft guide. One or more of the first shaft guide, second shaft guide and motor can be secured to the motor housing. In some embodiments the first shaft guide is secured to the housing and the motor and second shaft guide are free to move with respect thereto. A linear guide can be incorporated within the housing to guide the motor and the second shaft guide in the housing, and prevent the housing and the second shaft guide from rotating relative to the first shaft guide.

In some embodiments, at least one of the first motor shaft and the second motor shaft can have two or more starts. In addition or instead, the pitch of the first screw thread can be inverted from that of the second screw thread. At least one of the screw threads can be a ball screw thread or other type of screw thread. Where a ball screw thread is used, the respective shaft guide has a ball assembly.

According to some embodiments, a manipulator can have a segmented arm with two or more arm assemblies pivotally connected to one another. At least one of the two or more arm assemblies can include a linear motion actuator, a housing, and a pivot mechanism. The linear motion actuator can be any of the iterations just described. For example, the linear motion actuator can include a double-shafted motor, and first and second shaft guides. The double-shafted motor can comprise a first motor shaft having a first screw thread and a second motor shaft having a second screw thread. A pitch of the first screw thread can be different from that of the second screw thread. The first shaft guide can have a threaded hole which engages with the first screw thread. The second shaft guide can have a threaded hole which engages with the second screw thread. The pivot mechanism can be used to pivot an adjacent arm assembly of the segmented arm. The pivot mechanism can comprise a member mounted on the second shaft guide of the linear motion actuator and extending toward the adjacent arm assembly. The first shaft guide can be secured to the tubular housing with the motor and second shaft guide being movable within the housing relative to the first guide shaft.

In some embodiments, the member of the pivot mechanism can comprise a push rod and the adjacent arm assembly can have a protrusion engaged with the push rod. Alternatively, the member of the pivot mechanism can be pinned to the second shaft guide at a first end and to the adjacent arm assembly at a second end. The pivot mechanism may include a spring to bias the adjacent arm assembly to a first position. The manipulator may also include an angular encoder.

According to some embodiments, the adjacent arm assembly has a distal end configured to couple to an end effector. The end effector can be actuated by a second linear motion actuator in the adjacent arm assembly. The end the end effector can be removably coupled to the distal end of the adjacent arm assembly. The adjacent arm assembly may further comprise a rotating member configured to support an organ. The rotating member can be actuated by the second linear motion actuator in the distal arm assembly through a rack-and-pinion so as to rotate about an axis orthogonal to a longitudinal axis of a housing of the adjacent arm assembly.

According to some embodiments, a manipulator can have a segmented arm with a distal arm assembly and a proximal arm assembly pivotally connected to the distal arm assembly. The distal arm assembly can have a distal end configured to couple to an end effector. The proximal arm assembly can include a linear motion actuator, a housing, and a pivot mechanism. The linear motion actuator can be any of the iterations just described. For example, the linear motion actuator can include a double-shafted reduction drive comprising first and second reduction drive shafts. The first reduction drive shaft can have a first screw thread and the second reduction drive shaft can have a second screw thread. Each of the first and second screw threads are threaded on first and second spindles, respectively. The linear motion actuator may further include a first reduction drive shaft guide having a threaded hole which engages with the first screw thread and a second reduction drive shaft guide having a threaded hole which engages with the second screw thread. The pivot mechanism can be configured to pivot the distal arm assembly. The pivot mechanism can comprise a member mounted on the second shaft guide of the linear motion actuator and extending toward the distal arm assembly. The first shaft guide can be secured to the housing, and the motor and the second shaft guide are movable within the housing relative to the first guide shaft.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a partial cross-sectional view of a manipulator with linear motion actuators.

FIG. 1A is an end view of the manipulator of FIG. 1 showing the end effector and other features.

FIGS. 2A and 2B illustrate movement of a linear motion actuator.

FIG. 3 illustrates a conceptual diagram for a linear motion actuator having a high reduction ratio without reducing the pitch of the screw threads.

FIGS. 4A and 4B show movement of a manipulator with a linear motion actuator.

FIGS. 4C and 4D show cross-sectional views taken along lines 4C-4C and 4D-4D, respectively of FIG. 4B.

FIG. 5 shows another embodiment of manipulator.

FIGS. 6A and 6B are show movement of another embodiment of a manipulator.

FIGS. 6C and 6D are cross sectional views of the manipulator of FIGS. 6A and 6B, respectively.

FIG. 7 is an illustration of a manipulator being used to support a body part.

FIG. 8 shows another embodiment of manipulator.

FIGS. 9 and 10 illustrate a conventional manipulator.

FIGS. 11A and 11B show parts of a reduction drive in another conventional manipulator.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. While the following detailed description is related to manipulators and linear motion actuators that may be employed in a manipulator, it is to be appreciated that the concepts described herein are also applicable to many other devices and methods.

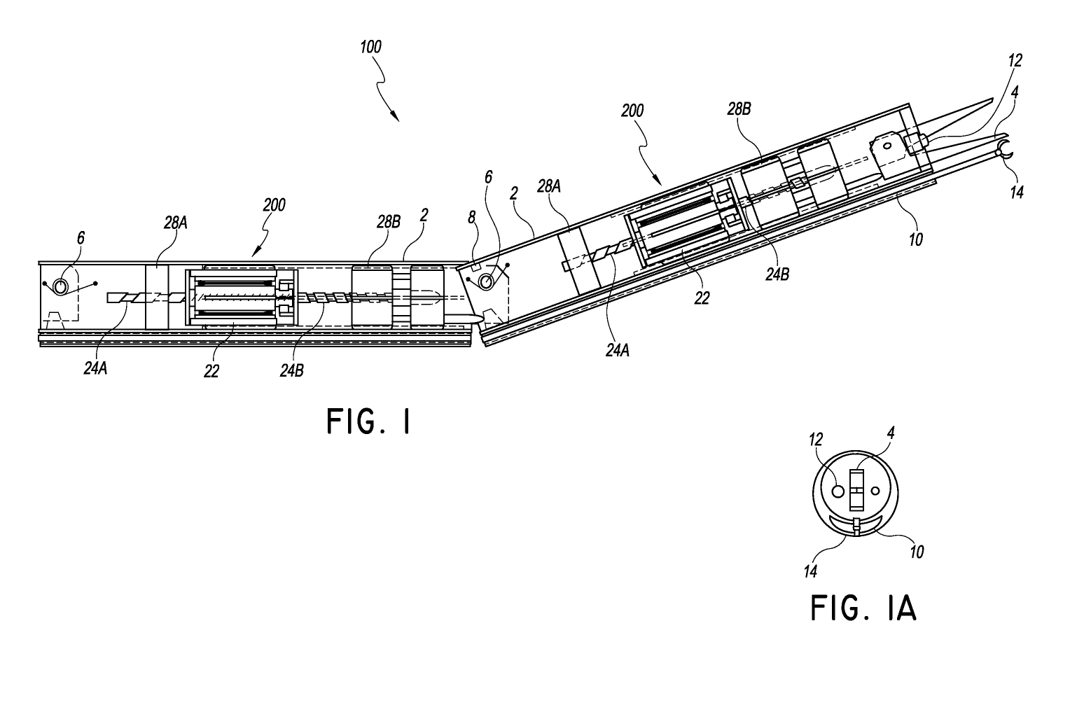

FIG. 1 illustrates one embodiment of a manipulator 100. The manipulator 100 is shown with two linear motion actuators 200, though it will be understood that embodiments of the manipulator 100 may have more or less linear motion actuators 200, and may in fact not have any linear motion actuators 200 but may include other types of systems to provide any necessary actuation. The manipulator 100 can be used in laparoscopic surgery or elsewhere.

A manipulator 100 can include an arm assembly 2 and an end effector 4. The end effector 4 can be any type of tool, for example in the surgery context, example end effectors can include, but are not limited to: probes, graspers, scissors, forceps, clip appliers, scalpels, electro cautery probes, and retractors. In other contexts, an end effector may include screwdrivers, suction, grabbers, hooks, etc. The manipulator may be stationary or moveable in position and can be positioned in such a way to allow use of the end effector 4 or other tool. The end effector 4 may be permanently or removably attached to the distal end of the manipulator.

In some embodiments, a manipulator 100 may include a segmented arm with two or more arm assemblies 2 connected to one another. The manipulator can be used to control the end effector 4, but also the relationship between the arm assemblies. For example, as will be described in more detail below, the two or more arm assemblies 2 can be connected pivotally, telescopically, etc. The illustrated embodiment of FIG. 1 shows a pivot 6 where the arm assemblies 2 are connected. An angular encoder 8 may also be included to monitor an angle between the arm assemblies 2.

The manipulator 100 can include any of a number of additional features. For example, FIG. 1 shows the manipulator 100 with a tubular housing having an instrument guide hole 10 and camera 12 (such as a CCD camera). A pair of operating forceps 14 is shown positioned within the instrument guide hole 10. Though it will be understood that any of a number of different tools could be advanced through the instrument guide hole 10.

An actuation mechanism as part of the manipulator 100 can control one or more of the end effector(s) 4, other tool(s), and the relationship between arm assemblies 2. The manipulator 100 as illustrated has two linear motion actuators 200. A linear motion actuator 200 can provide linear motion which the manipulator can use for linear movement or the linear motion can be converted into another type of motion such as rotational movement, scissor movement, etc. The linear motion actuator 200 can include a motor 22 having one or more shafts 24A, 24B. The shaft(s) can have a screw thread engaging a shaft guide 28A, 28B that can cause linear movement with rotation of the shaft. In the illustrated embodiment, the linear movement can cause movement of the arm assemblies and/or movement of the forceps being used as an end effector 4.

It will be appreciated that a screw thread (or thread) is a helical structure often used to translate rotational motion to linear motion, known in some instances as a lead-screw or power screw. A screw thread is generally in the form of a ridge extending around a cylinder or cone in the form of a helix. The mechanical advantage of a screw thread can depend on various factors including the screw thread's lead (the linear distance the screw travels in one revolution) and the pitch (the distance from the crest of one thread to the next). It is possible to obtain a higher torque by reducing the thread pitch. However, the fine threading which results from the reduction of the pitch may not have adequate mechanical strength to exert the desired (high) torque. Materials capable of fine threading to exert high torque are also limited and can add to the already higher manufacturing cost associated with fine threading.

In the case where several gears are used for a small manipulator, the gear teeth must be made extremely small during manufacture, and even in the case of a usual helical channel or screw thread, the helical channel pitch must be made very small to obtain a large reduction ratio. Accordingly, mechanical strength becomes a significant issue and leads to limitations in the selection of materials. However, as shown above, it is not necessary to make the helical channel pitch very small in order to obtain a reduction ratio by means of the pitches of the two channels, thus increasing the number of viable materials.

Types of screw thread can include, but are not limited to, triangular thread, square thread, trapezoidal thread, or ball thread. When a ball thread is used, the first shaft guide 28A and second shaft guide 28B can further comprise a ball assembly. In addition, the screw thread can include a multi-start thread, such as a two, three or more start thread which can change the lead of the screw.

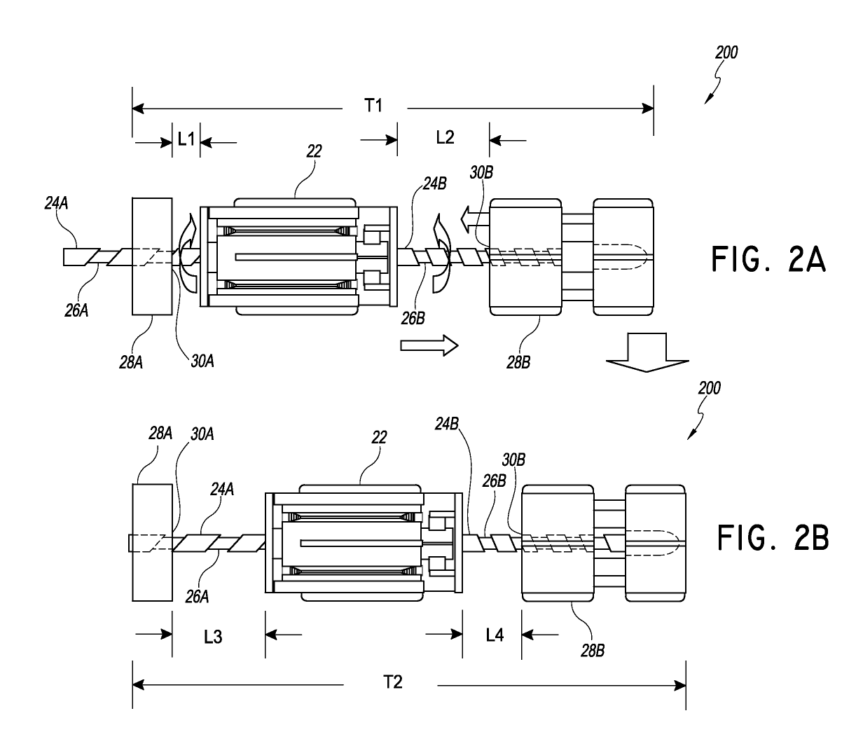

Turning now to FIGS. 2A and 2B, components and functioning of an embodiment of linear motion actuator 200 will be described. In the illustrated embodiment, two screw threads are shown which can allow the linear motion actuator 200 to provide high control and high torque without relying on expensive materials and fine threading, among other benefits. As has been mentioned, though the linear motion actuator 200 is described in the context of a manipulator 100 (illustrated in FIG. 1) it will be understood that the linear motion actuator can also be used in other types of devices and systems.

A linear motion actuator 200 can include a double-shafted motor 22 having a first motor shaft 24A with a first screw thread 26A and a second motor shaft 24B with a second screw thread 26B. A first shaft guide 28A can have a threaded hole 30A which engages with the first screw thread 24A and a second shaft guide 28B can have a threaded hole 30B which engages with the second screw thread 26B. One of the first shaft guide 28A, second shaft guide 28B, and motor 22 can be fixed in relation to the others so as to cause linear movement from rotation of the motor shafts.

In the illustrated example, the first shaft guide 28A is fixed in position with relation to both the second shaft guide 28B and the motor 22. Rotation of the motor shafts thereby causes one or both of the second shaft guide 28B and motor 22 to move away from or closer to the first shaft guide 28A. Though the screw threads can be the same, differences between them can be used to control the movement in a desired manner. For example, the pitch and/or lead of the first screw thread 26A can be different from that of the second screw thread 26B. In addition, the first screw thread 26A can be inverse to the second screw thread 26B (left-handed versus right-handed).

FIGS. 2A and 2B illustrate the change of position upon actuation of certain embodiments of linear motion actuator 200. In the illustrated example, actuation of the linear motion actuator 200 along the longitudinal axis is shown increasing the total length between the first motor guide 28A and the second motor guide 28B from a first length T1 to a second length T2. As the illustrated motor 22 moves together with the first motor shaft 24A, the distance between the first shaft guide 28A and the motor 22 increases from a first length L1 to a second length L3. At the same time, the second motor shaft 24B also rotates along the longitudinal axis. In the illustrated implementation, the first screw thread 26A is inverse to the second screw thread 26B (left-handed versus right-handed). Thus, upon actuation of the motor 22, the second shaft guide 28B moves along the second screw thread 26B in the opposite direction to the motor 22. As shown, the second shaft guide 28B can move closer to the motor 22 (FIG. 2B) such that a distance between the motor 22 and the second shaft guide 28B decreases from a first length L2 to a second length L4.

A net travel distance (or in this instance, increase in total length) (T2-T1) of the second shaft guide 28B with relation to the first shaft guide 28A can be obtained by calculating (L3-L1)-(L2-L4). The illustrated linear motion actuator 200 can beneficially provide a desired net travel distance of the second shaft guide 28B based on a number of rotations of the shafts. The respective pitch and/or lead of the first and second screw threads 26A, 26B can be set accordingly. For example, one can select a desired net travel distance of the second shaft guide 28B with respect to a number of rotations by selecting different pitches between the first screw thread 26A and the second screw thread 26B. In addition, the first screw thread 24A can be inverse to the second screw thread 24B. This can allow the linear motion actuator 200 to create an smaller overall linear movement without requiring fine threading. In addition, a large reduction ratio can be obtained, and it is possible to obtain a large torque with a small motor. If the screw threads are not inverse, than a larger overall linear movement can be made with finer threading and higher torque than a comparable single screw thread.

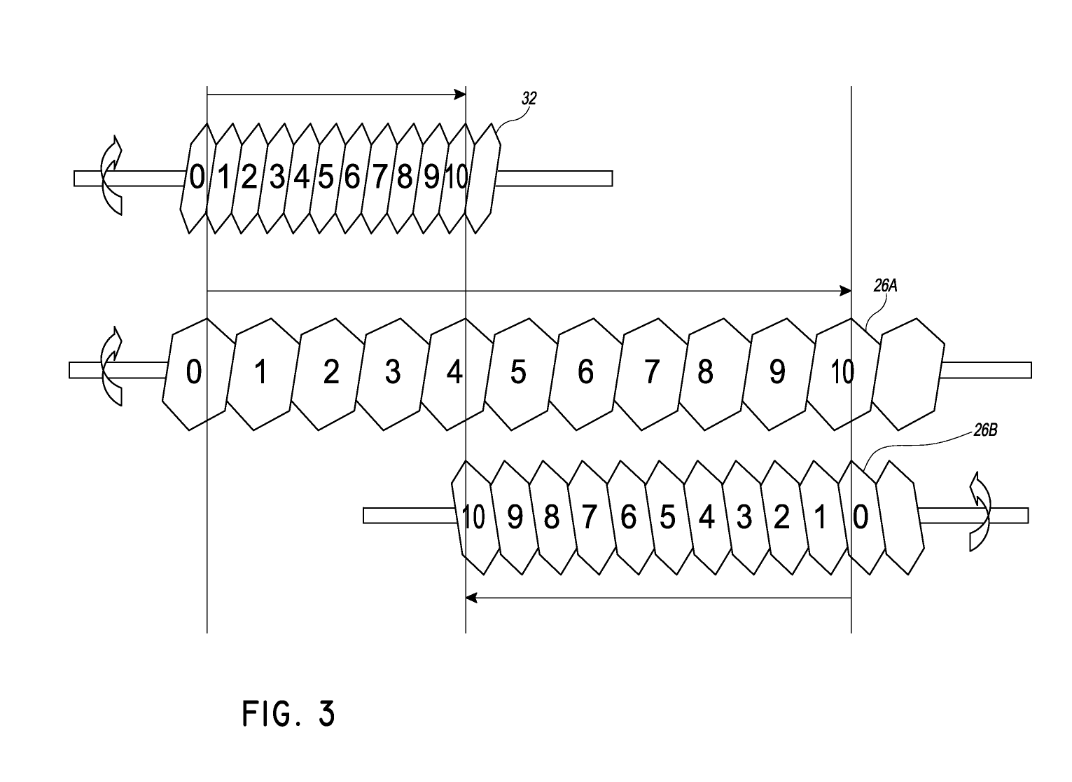

The conceptual diagram of FIG. 3 further illustrates certain aspects of the foregoing description. A fine pitch screw 32 is shown with a travel distance indicated for ten rotations. The first and second screw threads 24A, 24B are also shown including their respective travel distances based on ten rotations. The travel distance for fine pitch screw 32 is equivalent to that obtained by using two helical screws which travel in opposite directions and have the different pitches indicated. Advantageously, both the first and second screw threads 24A, 24B have pitches larger than that of the fine pitch screw 32, though this is not required. In some embodiments, the combination and orientation discussed herein increases the amount of torque per revolution so that a higher thrust can be obtained. The difference in pitch of the first screw thread 24A and the second screw thread 24B can allow the double shafted motor to maintain a large reduction ratio. The linear motion actuator 200 according to some embodiments is able to provide an increased mechanical advantage, without having to have the normally associated pitch of screw threads which may be more expensive and difficult to manufacture and maintain.

Returning to FIGS. 2A and 2B, it may be further appreciated that the components of the linear motion actuator 200 can work together to resist backdriving to thereby effectively lock the system in place when the motor is not activated. The first and second motor shafts 24A, 24B can be linked through the double-shafted motor 22. Thus, rotation of one shaft can result in an equal rotation of the other. When the motor shafts 24A, 24B are not rotating, a linear force on the second shaft guide 28B can apply torque on the second screw thread 26B and induce the secured first shaft guide 28A to apply torque on the first screw thread 24B in a direction opposite to that of the second screw thread 24A. Hence, a high resistance to backdriving can be induced and self-locking of the system can be achieved.

In some embodiments, the double-shafted motor 22 can further include one or more spindles (not shown). The spindles can be coupled to the first motor shaft 24A and/or the second motor shaft 24B. In such embodiments, flexible couplers may be used to couple the spindles to the motor shafts so that the alignment between the double-shaft motor 22, and the shaft guides 28A, 28B need not be tight. Accordingly, it will be appreciated that the manufacturing cost can be reduced. Rigid couplers may also be used. In some embodiments, a spindle can include the motor shaft, bearings, and/or other element attached to the motor shaft, such as a clamp or a chuck. It will also be understood that the double-shafted motor 22, first motor shaft 24A and second motor shaft 24B can be made of a single continuous rod protruding from both ends of the motor.

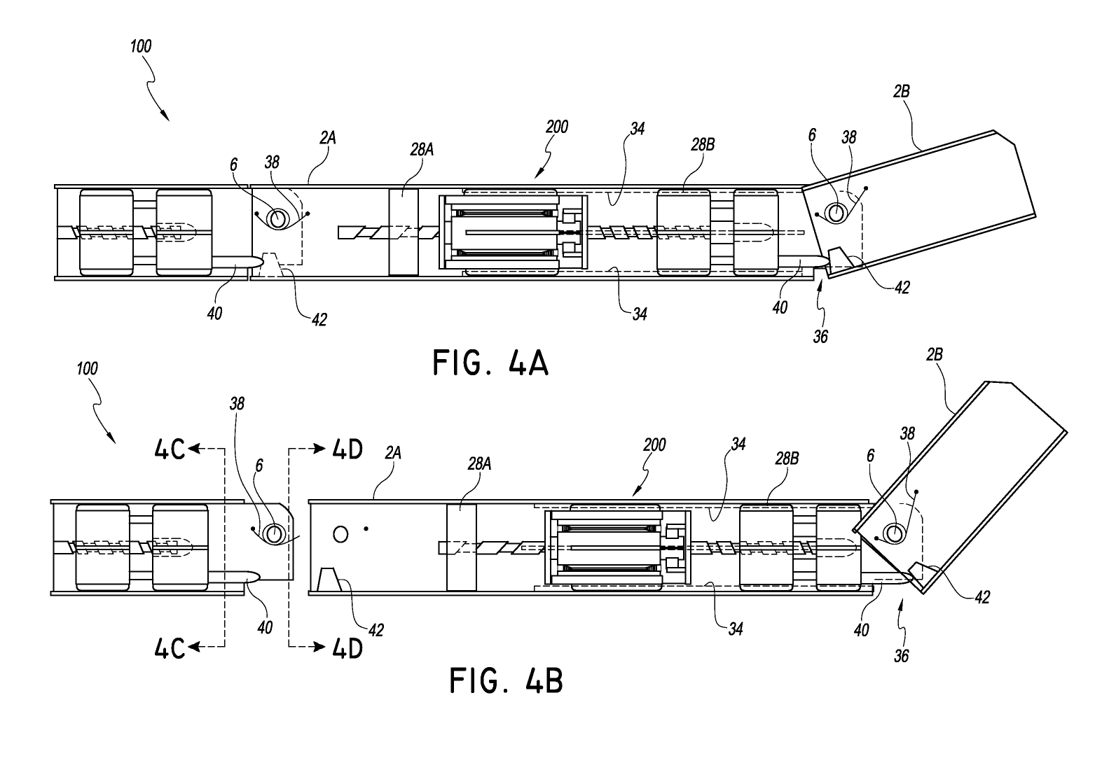

A linear motion actuator 200 can further include a housing to accommodate the double-shafted motor. In the illustrated embodiments, such in FIGS. 4A and 4B, an arm assembly 2A can form the housing. The housing is shown as a tubular, cylindrical housing, though other shapes can also be used. The shape may be circular, ellipse, square, hexagon, or the like. It is preferable to use titanium or stainless steel for the housing for medical applications such as a laparoscopic manipulator. A composite resin or other material may also be used.

In some embodiments, the housing can further include a linear guide 34 to facilitate linear movement of the motor 22, first shaft guide 28A and/second shaft guide 28B. The linear guide 34 can comprise a groove (FIG. 4C), rail, or rod fixed in position within the housing. One or more of the motor 22, first shaft guide 28A and second shaft guide 28B can slidably engage the linear guide 34. The linear guide 34 can also prevent rotation between the housing and components of the linear motion actuator 200. As illustrated, the arm assembly 2A has four linear guides 34, though any number of linear guides 34 can be used.

As shown in FIGS. 4A and 4B, the first shaft guide 28A is secured to the arm assembly or tubular housing 2A, while the second shaft guide 28B and the double-shafted motor 22 are movable along the longitudinal axis of the tubular housing. The linear guide 34 forms a rail along which the motor 22 and second shaft guide 28B can slide. The motor 22 and second shaft guide 28B can both include a corresponding groove, hole, pair of protrusions, etc. to engage the rail 34. Accordingly, the motor 22 and second shaft guide 28B can move in a linear motion without rotating within the housing.

By employing the linear motion actuator in accordance with the non-limiting embodiments described above, a manipulator for a laparoscope or other device having improved performance can be achieved. FIGS. 4A-8 show various embodiments of manipulators or portions thereof. For clarity, the figures and associated descriptions may on occasion show only two segments (also referred to as arm assemblies) of the manipulator, and a proximal and/or distal end may be omitted. It should be understood that the manipulator can include three or more segments, and the proximal end can be coupled to an interface of a robotic surgical system (not shown) using known methods.

FIGS. 4A and 4B show the actuation of a linear motion actuator 200 within a manipulator 100. As can be seen, the angle of the arm assemblies 2A, 2B is changed by movement of the linear motion actuator 200. The two arm assemblies 2A, 2B can be pivotally connected at pivot 6. The manipulator 100 can include a pivot mechanism 36 configured to pivot an adjacent arm assembly 2B of the segmented arm. The pivot mechanism can include a member 40 mounted on the second shaft guide 28B of the linear motion actuator 200 and extending toward the adjacent arm assembly 2B. The member 40 can be a push rod and the adjacent arm assembly 2b can include a protrusion 42 engaged with the push rod. The push rod 40 engages the protrusion 42 mounted in the adjacent arm assembly so as to move the adjacent arm assembly. A spring 38, such as a torsion spring, can be used as part of the pivot mechanism 36 to maintain the relationship of the arm assemblies in a desired position before or after movement of the linear motion actuator 200. FIG. 4D illustrates the position of the torsion spring 38 with respect to the pivot 6.

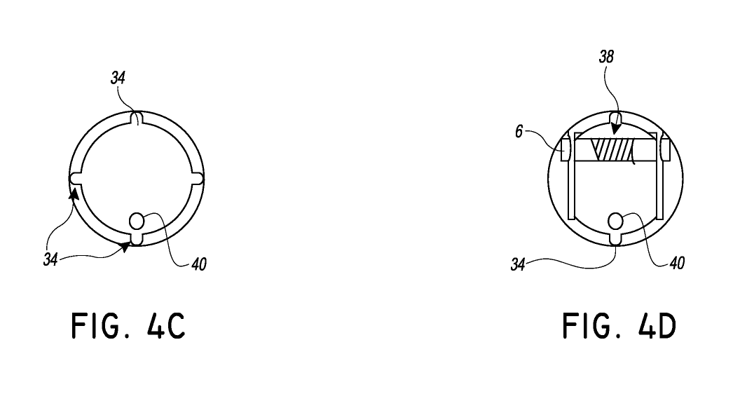

In some embodiments, the pivot mechanism 36 can include a linkage system, such as that shown in FIG. 5. The linkage system may pivot the adjacent arm assembly. The mechanism may comprise a member or link 44 pinned 46 to both arm assemblies at 48 and 50. The link 44 can be pinned to the second shaft guide 28B at a first end and to the adjacent arm assembly 2B at a second end. Bearings may also be used in at the pins to reduce friction. The linkage system may include a plurality of the links and joints. In addition, though a torsional spring is not shown, it may also be included to help maintain the relationship of the arm assemblies.

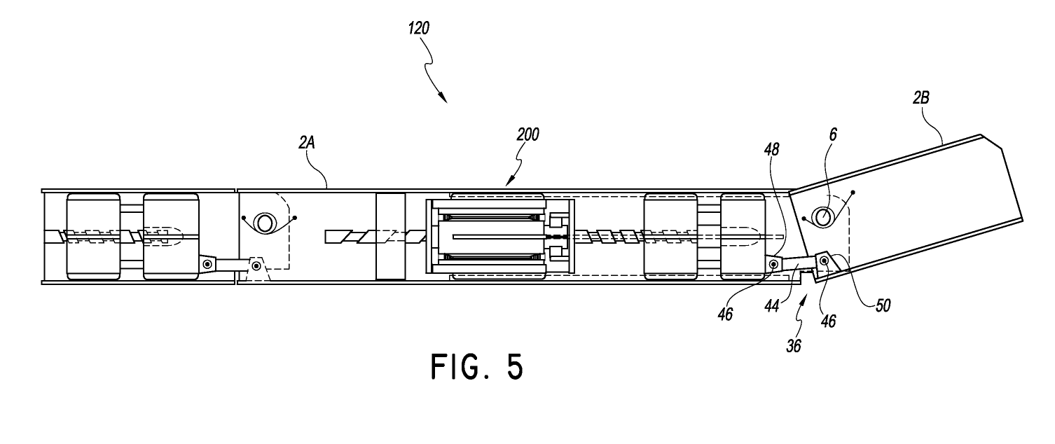

FIGS. 6A-D show another manipulator 130 in which the arm assembly 2 at the distal end further comprises a rotating member 52. The rotating member 52 can be used for one of many purposes including supporting an organ. The rotating member 52 can be actuated by the linear motion actuator 200 through a rack-and-pinion 54 so as to rotate about an axis orthogonal to a longitudinal axis of the tubular housing.

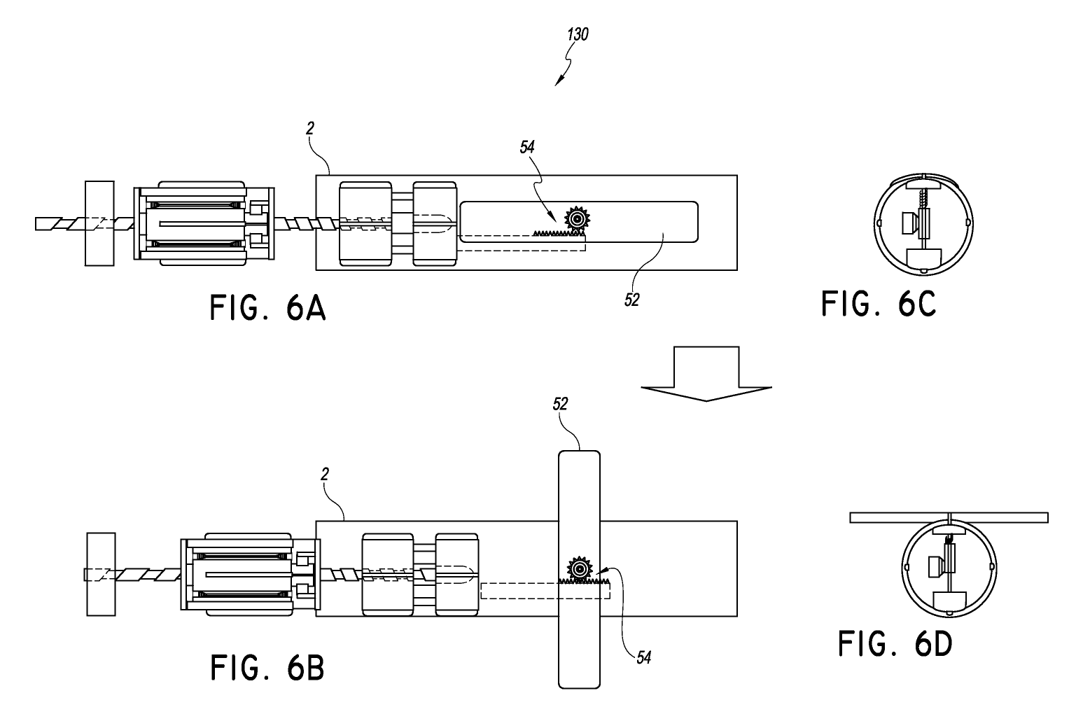

In FIG. 7, the manipulator 140 is shown where the rotating member 52 has been moved to the tip of one of the arm assemblies 2. This can be used for example, in situations where an internal organ 56 needs to be supported during a laparoscopic procedure. The internal organ 56 shown is a pancreas being supported from below. As the linear motion actuator 200 in accordance with non-limiting embodiments described above provides high resistance to a backdriving, the rotational member 52 can be locked in position to hold the organ 56, without using a separate locking mechanism.

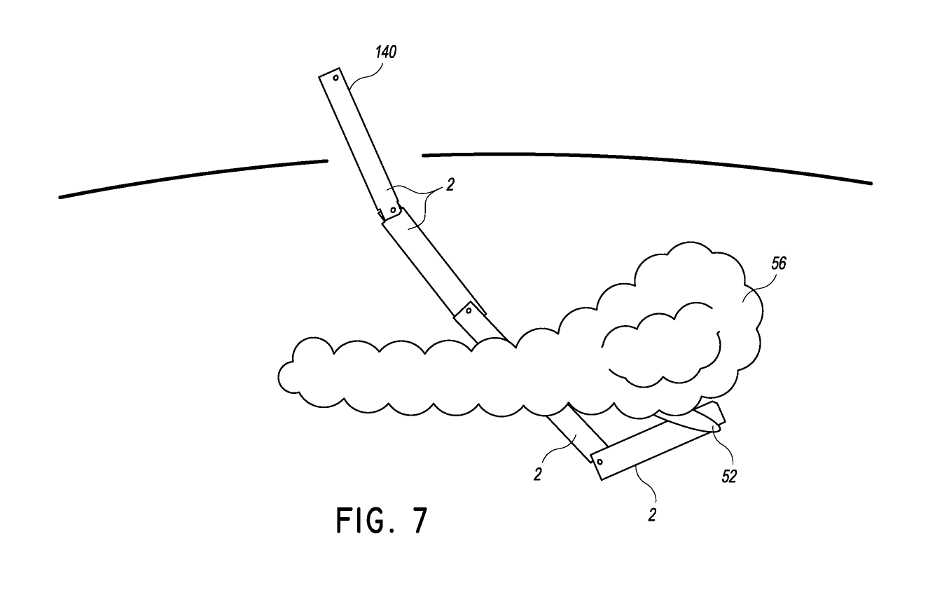

Turning now to FIG. 8, a manipulator 150 according to another embodiment is shown. In the manipulator 150 the double-shafted motor 22 of the linear motion actuator 200 has been replaced with a motor 22′ and a reduction drive assembly 58. The motor 22′ and reduction drive assembly 58 can increase the amount of torque per revolution so that a higher thrust can be obtained. A reduction drive assembly 58 can include a plurality of gears, such as a small driving gear 62 connected to the motor 22′ and a larger driven gear 60 connected to the double shaft 24. It will be understood that any number of different gear arraignments can be used to provide a reduction in speed between the motor and the double shaft 24. The double shaft 24 can include the first shaft 24A having the first screw thread 26A and the second shaft 24B having the second screw thread 26A. The double shaft 24 may include a single rod having two different threads, but preferably is made from two separate rods that are linked to together. It can be easier to manufacture two rods with different threads than to manufacture a single rod with two different threads to form the double shaft. Rigid or flexible coupler may be used to connect the first shaft 24A and the second shaft 24B.

In the illustrated linear motion actuator 200′, it can be seen that the double shaft 24 is not in the center of the arm assembly. It should be understood that all of the linear motion actuators 200, 200′ discussed herein can be centered or off-center within their respective housings.

Many of the conventional laparoscopic manipulators use one or more of a plurality of cables, a reduction drive, or a miniature motor to achieve controlled movement of the manipulator arms and/or end effectors.

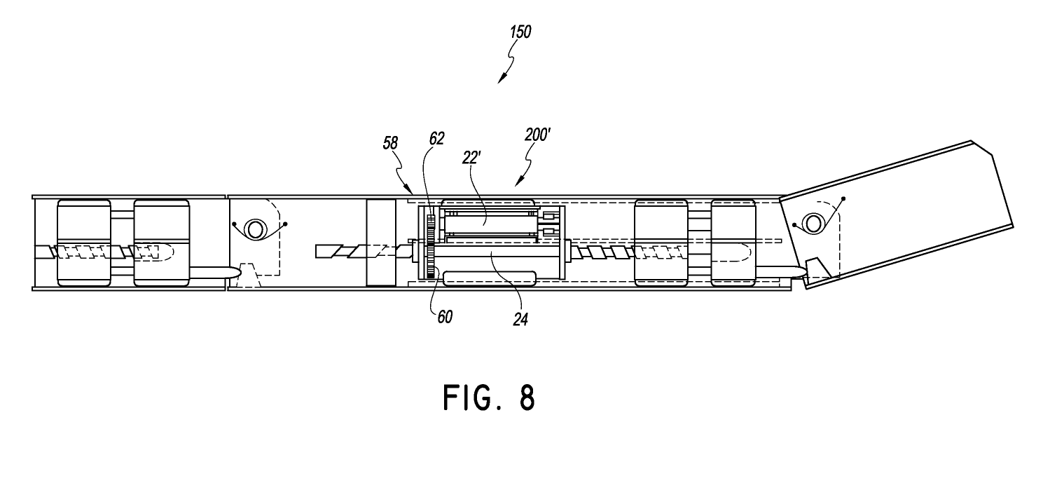

In situations in which the target sites for laparoscopic procedures are performed on the reverse side of an internal organ, the manipulator arm(s) may need to re-position and/or support the weight of the internal organ—thereby facilitating access to the target site. FIG. 9 is a schematic diagram of an existing manipulator 160. In the manipulator 160, wires 64 are used to change the angle of the arm assemblies. Stretching of the wires occurs when a load is applied. Therefore, the wires may require frequent adjustment. Moreover, there are cases in which the wires have snapped during a procedure because it cannot withstand the weight of the internal organ.

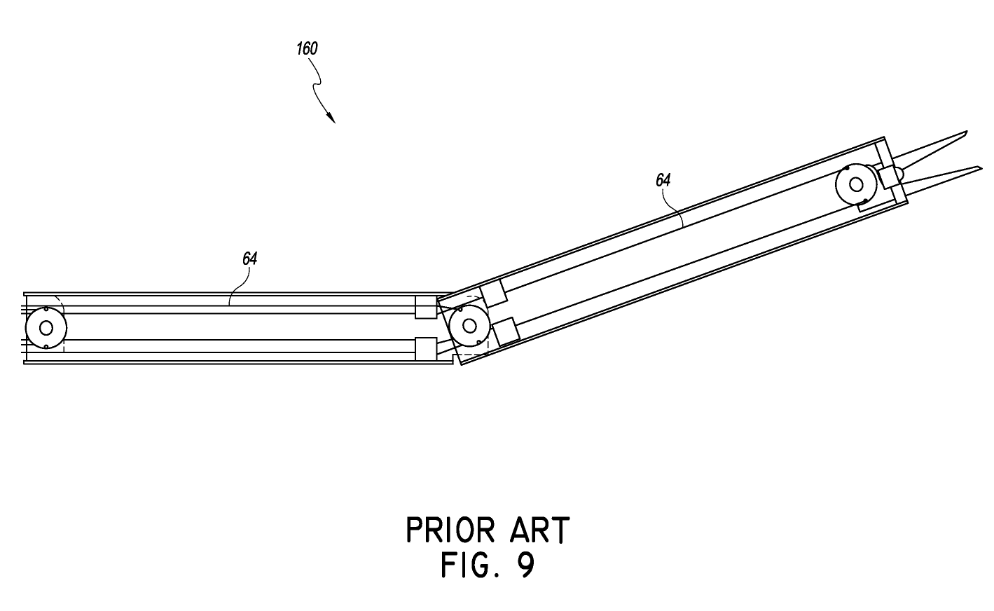

Another problem encountered with conventional laparoscopic manipulators 170 relates to the use of a miniature motor 72 (FIG. 10) to generate movement. These miniature motors 72 alone generally do not provide enough torque to effectively operate the manipulator 170. Accordingly, reduction drives 66 are also needed. Reduction drives 66 are used to reduce rotational speed and increase the amount of torque per revolution of a shaft. Typically, a multiple gears are used in these reduction drives to provide the desired reduction ratio. The more gears used may increase the likelihood of “backlash” and induce a waggling motion on the manipulator arms. Although reduction drives using a number of gears to provide the desired high reduction ratio can be made to avoid “backlash”, such drives are typically too bulky for use in laparoscopic manipulators. Further, high-precision machining is required for these improved reduction drives increasing the manufacturing cost.

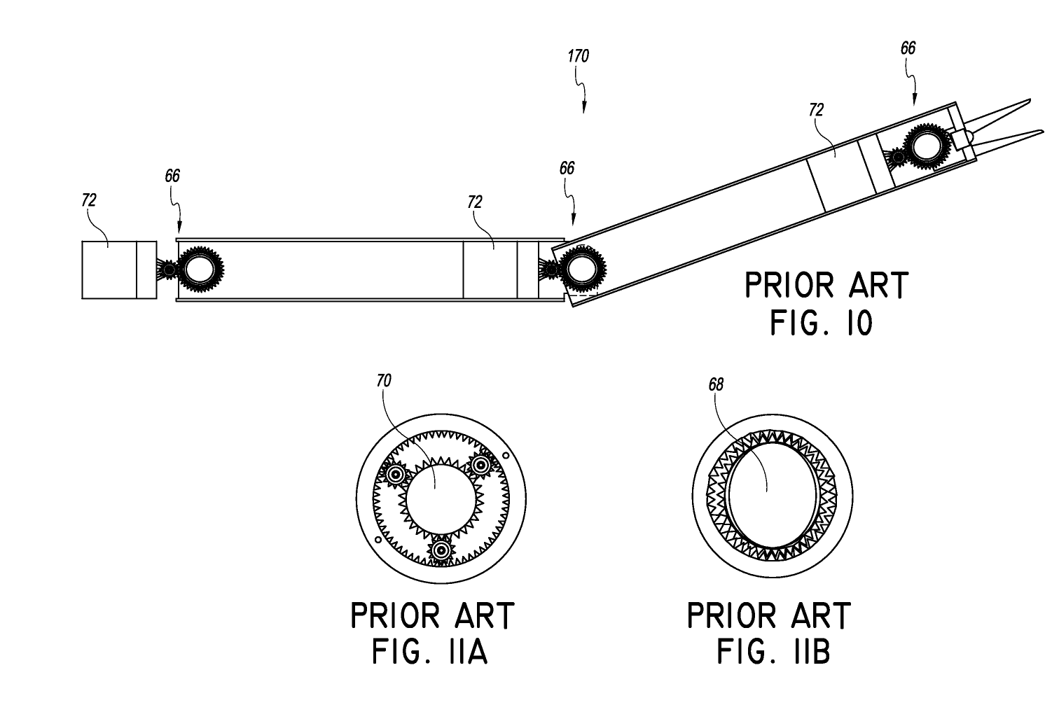

One solution to the reduction ratio and “backlash” issues has been the use of a planetary reduction drive 70 as seen in FIG. 11A. However, if a planetary gear is used, it is necessary to prepare a large number of reduction gears, and for this reason vibrations occur due to backlash, making it difficult to perform delicate work. Furthermore, although it is possible to use harmonic drive gearing to suppress backlash, additional torque is required from the motor because it is necessary to deform the flex spline 68 into an ellipse (FIG. 11B). Consequently, a high torque motor and/or another reduction drive are required to utilize the harmonic drive gears. Thus, there remains a need for a solution to the reduction ratio and “backlash” issues, which are hampering development of effective, low profile and affordable laparoscopic manipulators.

The manipulators and linear motion actuators as described herein provide many benefits and advantages over the conventional devices. For example, sufficient torque to drive the manipulator arm can be obtained even when using a small motor. The number of reduction gears can be reduced, and a high-precision manipulator can be realized. A large load can be supported with a simple structure as the disclosed linear motion actuator can naturally resist backdriving. Manufacturing costs can also be controlled because the number of components is substantially reduced and the need for high tolerances, such as with fine threading, is also reduced.

The helical shaft system of the linear motion actuator can provide a greater reduction ratio with fewer parts than the typical circular gear based systems. For example, this can be done by providing two helical channels which are inverted from one another and on the same axis. In addition, a difference in pitch between the helical channels can be used to provide greater control of the linear movement to be converted into radial or other types of movement to be used by a manipulator.

As detailed above, a small manipulator can be equipped with a high-torque running gear, allowing delicate operations. It can be used in laparoscopies and will contribute to the development of medical apparatuses with lower production costs. Two types of helical gear can be used to achieve large reduction ratio. Precise angle setting is possible. A large reduction ratio can be achieved with few precision parts. Large loads can be supported because of the few precision gears. In addition, if a larger reduction ratio is needed, a motor with a reduction gear can be used.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A linear motion actuator, the linear motion actuator comprising:
a double- shafted motor comprising a first motor shaft having a first screw thread and a second motor shaft having a second screw thread, wherein a pitch of the first screw thread is different from that of the second screw thread;
a first shaft guide having a threaded hole which engages with the first screw thread;
a second shaft guide having a threaded hole which engages with the second screw thread;
a housing to accommodate the motor, the first shaft guide, and the second shaft guide; and
a linear guide configured to guide the motor and the second shaft guide in the housing, and prevent the housing and the second shaft guide from rotating relative to the first shaft guide.

2. The linear motion actuator of claim 1, wherein the first shaft guide is secured to the housing.

3. (canceled)

4. The linear motion actuator of claim 1, wherein at least one of the first motor shaft and the second motor shaft has two or more starts.

5. The linear motion actuator of claim 1, wherein at least one of the first screw thread is a ball screw thread and the first shaft guide comprises a ball assembly or the second screw thread is a ball screw thread and the second shaft guide comprises a ball assembly.

6. The linear motion actuator of claim 1, further comprising a first spindle coupled to the first motor shaft or the second motor shaft.

7. The linear motion actuator of claim 1, wherein the pitch of the first screw thread is inverted from that of the second screw thread.

8. A manipulator, the manipulator comprising:
a segmented arm comprising two or more arm assemblies pivotally connected to one another, and wherein at least one of the two or more arm assemblies comprises:
a linear motion actuator comprising:
a double-shafted motor comprising a first motor shaft having a first screw thread and a second motor shaft having a second screw thread, wherein a pitch of the first screw thread is different from that of the second screw thread;
a first shaft guide having a threaded hole which engages with the first screw thread; and
a second shaft guide having a threaded hole which engages with the second screw thread;
a housing;
a pivot mechanism configured to pivot an adjacent arm assembly of the segmented arm, the pivot mechanism comprising a member mounted on the second shaft guide of the linear motion actuator and extending toward the adjacent arm assembly,
wherein the first shaft guide is secured to the housing, and the motor and the second shaft guide are movable within the housing relative to the first guide shaft.

9. The manipulator of claim 8, wherein the pitch of the first screw thread is inverted from that of the second screw thread such that the motor and the second shaft guide are movable in opposite directions.

10. The manipulator of claim 8, wherein the first shaft guide is secured such that the second shaft guide is movable in an axial direction along a longitudinal axis of the housing, further comprising a linear guide configured to guide the motor and the second shaft guide along the longitudinal axis, and prevent the motor and the second shaft guide from rotating relative to the first shaft guide and the housing.

11. (canceled)

12. The manipulator of claim 8, wherein the member of the pivot mechanism comprises a push rod and the adjacent arm assembly includes a protrusion engaged with the push rod.

13. The manipulator of claim 8, wherein the member of the pivot mechanism is pinned to the second shaft guide at a first end and to the adjacent arm assembly at a second end.

14. The manipulator of claim 8, wherein:
the adjacent arm assembly has a distal end configured to couple to an end effector; and
the end effector is removably coupled to the distal end of the adjacent arm assembly.

15. The manipulator of claim 8, wherein:
the adjacent arm assembly has a distal end configured to couple to an end effector;
the end effector is actuated by a second linear motion actuator in the adjacent arm assembly; and
the adjacent arm assembly further comprises a rotating member configured to support an organ, wherein the rotating member is actuated by the second linear motion actuator in the distal arm assembly through a rack-and-pinion so as to rotate about an axis orthogonal to a longitudinal axis of a housing of the adjacent arm assembly.

16-17. (canceled)

18. The manipulator of claim 8, wherein at least one of:
the housing further comprises an instrument guide channel;
the pivot mechanism further comprises a spring to bias the adjacent arm assembly to a first position; or
the manipulator further comprises an angular encoder.

19-20. (canceled)

21. A manipulator for a laparoscope, the manipulator comprising:
a segmented arm comprising:
a distal arm assembly with a distal end configured to couple to an end effector; and
a proximal arm assembly pivotally connected to the distal arm assembly, the proximal arm assembly comprising:
a linear motion actuator comprising:
a double-shafted reduction drive comprising first and second reduction drive shafts, wherein the first reduction drive shaft has a first screw thread and the second reduction drive shaft has a second screw thread, wherein each of the first and second screw threads are threaded on first and second spindles, respectively;
a first shaft guide having a threaded hole which engages with the first screw thread; and
a second shaft guide having a threaded hole which engages with the second screw thread;
a housing;
a pivot mechanism configured to pivot the distal arm assembly comprising a member mounted on the second shaft guide of the linear motion actuator and extending toward the distal arm assembly,
wherein the first shaft guide is secured to the housing, and the motor and the second shaft guide are movable within the housing relative to the first guide shaft.

22. The manipulator of claim 21, wherein the pitch of the first screw thread is inverted from that of the second screw thread such that the motor and the second shaft guide are movable in opposite directions.

23. The manipulator of claim 21, wherein the first shaft guide is secured such that the second shaft guide is movable in an axial direction along a longitudinal axis of the housing.

24. The manipulator of claim 21, wherein the member of the pivot mechanism comprises a push rod and the distal arm assembly includes a protrusion engaged with the push rod.

25. The manipulator of claim 21, wherein the member of the pivot mechanism is pinned to the second shaft guide at a first end and to the distal arm assembly at a second end.

26. The manipulator of claim 21, wherein the end effector is actuated by a second linear motion actuator in the distal arm assembly.

27. The manipulator of claim 21, wherein the distal arm assembly further comprises a rotating member configured to support an organ, wherein the rotating member is actuated by a second linear motion actuator in the distal arm assembly through a rack-and-pinion so as to rotate about an axis orthogonal to a longitudinal axis of the housing.

28. The manipulator of claim 21, wherein at least one of:
the end effector is removably coupled to the distal end of the distal arm assembly;
the housing further comprises an instrument guide channel;
the pivot mechanism further comprises a spring to bias the distal arm assembly to a first position; or
the manipulator further comprises an angular encoder.

29-31. (canceled)
